(19) United States
(12) Patent Application Publication
(10) Pub. No.: US 2025/0288227 A1
(43) Pub. Date: Sep. 18, 2025

(54) STACKABLE MULTI ELECTRODE SENSOR ARRAY PROVIDING MODULARITY IN MAKING MULTIANALYTE SENSING DEVICES

(71) Applicant: Medtronic MiniMed, Inc., Northridge, CA (US)

(72) Inventors: Anna M. Tycon, Scottsdale, AZ (US); Omid Mahdavi, Fountain Hills, AZ (US); Mohsen Askarinya, Chandler, AZ (US); David A. Ruben, Mesa, AZ (US); Randal C. Schulhauser, Phoenix, AZ (US)

(21) Appl. No.: 19/075,849

(22) Filed: Mar. 11, 2025

Related U.S. Application Data
(60) Provisional application No. 63/565,760, filed on Mar. 15, 2024.

(51) Int. Cl. A61B 5/145; A61B 5/00; A61B 5/1486

(52) U.S. Cl. CPC A61B 5/14542; A61B 5/1486; A61B 5/742

(57) ABSTRACT

A multi-electrode sensor array includes a first electrode configured for sensing a first analyte, and a second electrode configured for sensing a second analyte. The first electrode includes a first electrical connection pad on a first end portion of the first electrode, and a first enzyme on a second portion of the first electrode, the first enzyme configured for sensing the first analyte. The second electrode includes a second electrical connection pad on a first end portion of the second electrode, and a second enzyme on a second portion of the second electrode, the second enzyme configured for sensing the second analyte.

### CROSS-REFERENCE TO RELATED APPLICATIONS

The present application claims the benefit of and priority to U.S. Provisional Patent Application No. 63/565,760, filed on Mar. 15, 2024, the entire contents of which is hereby incorporated by reference.

### FIELD

The present disclosure relates generally to continuous glucose monitoring (CGM) and more particularly to stackable multi-electrode analyte sensor arrays for providing modularity in multianalyte sensing devices.

### BACKGROUND

Analyte sensors such as biosensors include devices that use biological elements to convert a chemical analyte in a matrix into a detectable signal. There are many types of biosensors used for a wide variety of analytes, including amperometric glucose sensors for glucose level control for diabetes.

A typical glucose sensor works according to the following chemical reactions:

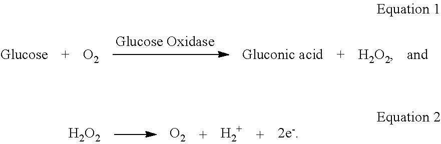

The glucose oxidase is used to catalyze the reaction between glucose and oxygen to yield gluconic acid and hydrogen peroxide, H₂O₂, (Equation 1). The hydrogen peroxide reacts electrochemically, as shown in Equation 2, and the current can be measured by a potentiostat. These reactions, which occur in a variety of oxidoreductases known in the art, are used in a number of sensor designs.

One common problem with analyte sensors is that, currently, fabricating a single sensor for a single analyte requires many wafer-scale chemical processing steps, making sensor fabrication expensive and complex. Multiple analyte sensors on a single wafer will require even more processing steps for a single wafer, increasing complexity and cost for fabrication while decreasing overall sensor yield per wafer.

There is room for improvement in the design of analyte sensors to reduce manufacturing steps that require incompatible materials and reduce expensive or labor-intensive steps.

### SUMMARY

The present disclosure relates generally to continuous glucose monitoring (CGM) and, more particularly, to stackable analytic sensor arrays for providing modularity in multianalyte sensing devices.

In accordance with aspects of the present disclosure, a multi-electrode sensor array includes a first electrode configured for sensing a first analyte and a second electrode configured for sensing a second analyte. The first electrode includes: a first surface of the first electrode which includes a first electrical connection pad on a first end portion of the first electrode; and a first sensing region on a second portion of the first electrode. The first sensing region is configured for sensing the first analyte and may include one or more enzymes or ionophores. The second electrode includes: a first surface of the second electrode which includes a second electrical connection pad on a first end portion of the second electrode; and a second sensing region on a second portion of the second electrode. The second sensing region is configured for sensing the second analyte.

In an aspect of the present disclosure, the first analyte may be different from the second analyte.

In another aspect of the present disclosure, the second electrode may be bonded to a second surface of the first electrode.

In yet another aspect of the present disclosure, the first enzyme may include a first analyte sensing molecule.

In a further aspect of the present disclosure, the first electrode may be electrografted or electropolymerized with the first analyte sensing molecule.

In yet a further aspect of the present disclosure, the first and second electrodes may each be configured to generate a detectable electrical signal upon exposure to the respective first and second analyte.

In an aspect of the present disclosure, the first electrode and the second electrode may each be formed by incompatible manufacturing processes.

In another aspect of the present disclosure, the multi-electrode sensor array may further include a third electrode configured for sensing a third analyte. The third electrode may include a first surface of the third electrode which includes a third electrical connection pad on a first end portion of the third electrode, and a third sensing region on a second portion of the third electrode. The third enzyme may be configured for sensing the third analyte.

In yet another aspect of the present disclosure, the third analyte may be different from the first and second analytes.

In yet another aspect of the present disclosure, a second surface of the second electrode may be bonded to a second surface of the third electrode.

In a further aspect of the present disclosure, the first end portion of the second electrode and the second end portion of the second electrode may extend past the first end portion of the first electrode and the second end portion of the first electrode, respectively.

In a further aspect of the present disclosure, the first electrode may further include: a third electrical connection pad on the first end portion of the first electrode; and a third enzyme on a second portion of the first electrode. The third enzyme may be configured for sensing a third analyte.

In accordance with aspects of the disclosure, a method of manufacturing a multi-electrode analyte sensor array is presented. The method includes processing a plurality of substrates of individual electrode types, including a first electrode and a second electrode from different substrates. The first electrode is configured for sensing a first analyte. A first surface of the first electrode includes a first electrical connection pad on a first end portion of the first electrode, and a first enzyme on a second portion of the first electrode. The first enzyme is configured for sensing the first analyte. The second electrode is configured for sensing a second analyte. A first surface of the second electrode includes a second electrical connection pad on a first end portion of the second electrode and a second enzyme on a second portion of the second electrode. The second enzyme is configured for sensing the second analyte.

In yet a further aspect of the present disclosure, the method may further include: stacking aligned substrates of the plurality of substrates; bonding the stacked aligned substrates to form stacked sensors; and separating individual stacked sensors from each of the bonded substrates to form the multi-electrode analyte sensor array.

In an aspect of the present disclosure, the method may further include: singulating each wafer of the plurality of substrates into single electrodes; stacking the single electrodes of at least two different types of electrodes; and bonding the stacked electrodes to form the multi-electrode analyte sensor array. The multi-electrode analyte sensor array may be a flex stack array.

In yet a further aspect of the present disclosure, the plurality of substrates of individual electrode types may further include a third electrode configured for sensing a third analyte. A first surface of the third electrode may include a third electrical connection pad on a first end portion of the third electrode and a third enzyme on a second portion of the third electrode. The third enzyme may be configured for sensing the third analyte.

In an aspect of the present disclosure, each of the plurality of substrates may include only the same electrode type on that wafer.

In yet another aspect of the present disclosure, the plurality of substrates may include at least two or more different electrode types.

In a further aspect of the present disclosure, the method may further include stacking a third electrode to the multi-electrode analyte sensor array. The third electrode may be configured for sensing a third analyte. A first surface of the third electrode may include a third electrical connection pad on a first end portion of the third electrode and a third enzyme on a second portion of the third electrode. The third enzyme may be configured for sensing the third analyte.

In accordance with aspects of the disclosure, a system for sensing multiple analytes includes: a multi-electrode sensor array; a processor wherein the first electrode and second electrode are communicatively coupled to the processor; and a memory.

The multi-electrode sensor array includes: a first electrode configured for sensing a first analyte; and a second electrode configured for sensing a second analyte.

The first surface of the first electrode includes a first electrical connection pad on a first end portion of the first electrode and a first enzyme on a second portion of the first electrode. The first enzyme is configured for sensing the first analyte. A first surface of the second electrode includes a second electrical connection pad on a first end portion of the second electrode and a second enzyme on a second portion of the second electrode. The second enzyme is configured for sensing the second analyte.

The memory includes instructions stored thereon which, when executed by the processor, cause the system to: obtain a respective signal from each of the first electrode and the second electrode; determine a value for the first analyte based on the signal from the first electrode; determine a value for the second analyte based on the signal from the second electrode; and display on a display the determined values for the first analyte and the second analyte.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of aspects of the disclosure will be made with reference to the accompanying drawings, wherein like numerals designate corresponding parts in the figures.

FIG. 1 illustrates a perspective view of a subcutaneous sensor insertion set and a block diagram of an analyte sensor electronics device, in accordance with one or more aspects of this disclosure;

FIG. 2 illustrates a cross-sectional side view of a multi-electrode sensor array of the sensor insert set of FIG. 1, in accordance with one or more aspects of this disclosure;

FIG. 3 illustrates a top view of individual sensors of the multi-electrode sensor array of FIG. 2, in accordance with one or more aspects of this disclosure;

FIGS. 4A-4C illustrate an alternative aspect, including an example six-electrode multi-electrode sensor array of the sensor insert set of FIG. 1, in accordance with this disclosure;

FIG. 5A illustrates an alternative aspect, including an example four-electrode multi-electrode sensor array of the sensor insert set of FIG. 1, in accordance with this disclosure;

FIG. 5B illustrates a cross-sectional side view of the four-electrode multi-electrode sensor array of FIG. 5A, in accordance with this disclosure;

FIG. 6A illustrates an alternative aspect, including an example five-electrode multi-electrode sensor array of the sensor insert set of FIG. 1, in accordance with one or more aspects;

FIG. 6B illustrates a cross-sectional side view of the five-electrode multi-electrode sensor array of FIG. 6A, in accordance with this disclosure;

FIG. 7 is a flow diagram of a computer-implemented method using the multi-electrode sensor array of FIG. 2, in accordance with aspects of this disclosure; and

FIG. 8 is a flow diagram of a method of manufacturing the multi-electrode sensor array of FIG. 2, in accordance with aspects of this disclosure.

### DETAILED DESCRIPTION

In the following description, reference is made to the accompanying drawings which form a part hereof and which illustrate several aspects of the present disclosure. It is understood that other aspects may be utilized, and structural and operational changes may be made without departing from the scope of the present disclosure.

The aspects herein are described below with reference to flowchart illustrations of methods, systems, devices, apparatus, and programming and computer program products. It will be understood that each block of the flowchart illustrations, and combinations of blocks in the flowchart illustrations, can be implemented by programming instructions, including computer program instructions (as can any menu screens described in the figures). These computer program instructions may be loaded onto a computer or other programmable data processing apparatus (such as a controller, microcontroller, or processor in a sensor electronics device) to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks, and/or menus presented herein. Programming instructions may also be stored in and/or implemented via electronic circuitry, including integrated circuits (ICs) and Application Specific Integrated Circuits (ASICs) used in conjunction with sensor devices, apparatuses, and systems.

The disclosed implantable analyte sensor enables individual electrode wafer-level processing to avoid incompatible processes when fabricating a single multi-electrode device. For example, for the disclosed multianalyte sensor for the detection of glucose, potassium, and creatinine for patients with AKI, there may be incompatibilities between the temperatures and solvents used in the preparation of the potassium sensor and the enzyme layer in the glucose sensor. For example, these temperatures and solvents will inactivate the enzyme GOx.

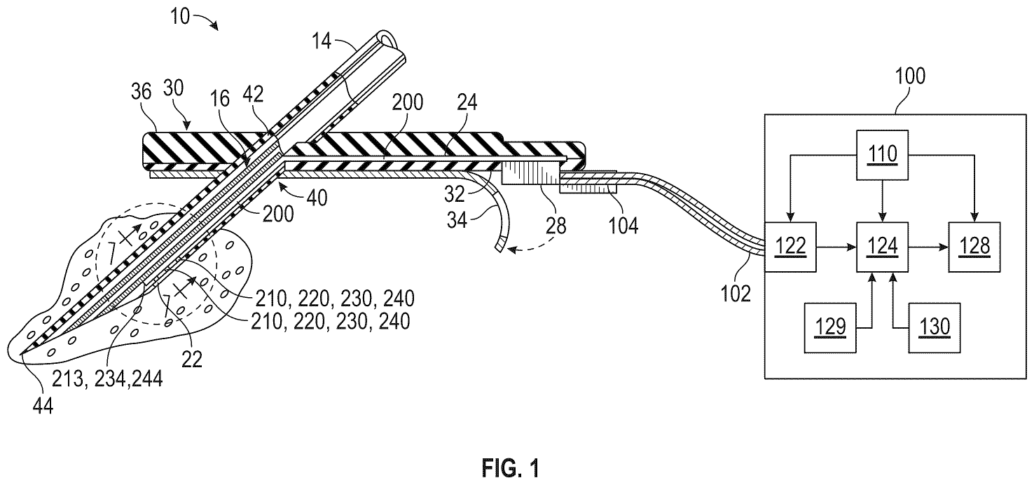

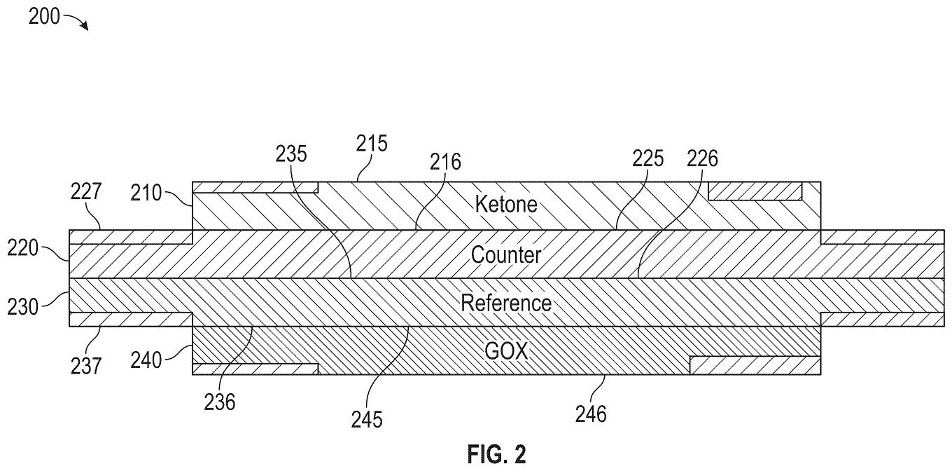

FIG. 1 is a perspective view of a subcutaneous sensor insertion set and a block diagram of a sensor electronics device according to various aspects of the disclosure. As illustrated in FIG. 1, a subcutaneous sensor set 10 is provided for subcutaneous placement of an active portion of an analyte sensor, such as multi-electrode sensor array 200 (see, e.g., FIG. 2), or the like, at a selected site in the body of a user. The subcutaneous or percutaneous portion of the sensor set 10 includes a hollow, slotted insertion needle 14, and a cannula 16. The needle 14 is used to facilitate quick and easy subcutaneous placement of the cannula 16 at the subcutaneous insertion site. Inside the cannula 16 is a sensing portion 214, 234, 244 of the analyte sensor 200 (e.g., a multi-electrode sensor array) to expose two or more sensor electrodes 210, 220, 230, 240 (FIG. 2) to the user's bodily fluids through a window 22 formed in the cannula 16. In an aspect of the disclosure, the two or more electrodes 210, 220, 230, 240 may include a counter electrode, a reference electrode, and two or more working electrodes. After insertion, the insertion needle 14 is withdrawn to leave the cannula 16 with the sensing portion 214, 234, 244 and the sensor electrodes 210, 220, 230, 240 in place at the selected insertion site. Although enzymatic sensors using analytes are used as an example, sensors configured for measuring non-analytes are contemplated to be within the scope of the disclosure. For example, the sensors may be mechanical and/or electrical such as piezo for pressure or thermocouples for temperature.

In particular aspects, the subcutaneous sensor set 10 facilitates accurate placement of a flexible thin film electrochemical analyte sensor 200 of the type used for monitoring specific blood parameters representative of a user's condition. The analyte sensor 200 monitors glucose levels in the body and may be used in conjunction with automated or semi-automated medication infusion pumps of the external or implantable type as described, e.g., in U.S. Pat. Nos. 4,562,751; 4,678,408; 4,685,903 or 4,573,994, the entire contents of which are incorporated herein by reference, to control delivery of insulin to a diabetic patient.

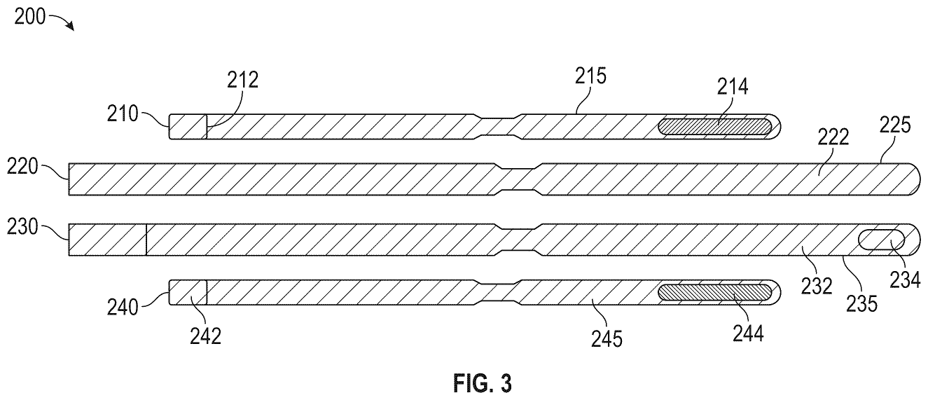

Particular aspects of the flexible analyte sensor 200 are constructed in accordance with thin film mask techniques to include elongated thin film conductors embedded or encased between layers of a selected insulative material such as polyimide film or sheet, and membranes. The sensor electrodes 210, 220, 230, 240 at a tip end of the sensing portion 214, 234, 244 are exposed through one of the insulative layers for direct contact with patient blood or other body fluids, when the sensing portion 214, 234, 244 (i.e., the active portion) of the analyte sensor 200 is subcutaneously placed at an insertion site. The sensing portion 214, 234, 244 is joined to an electrical connection pad 212, 222, 232, 242 (FIG. 3) that terminates in conductive contact pads, or the like, which are also exposed through one of the insulative layers. In alternative aspects, other types of implantable sensors, such as chemical based, optical based, or the like, may be used.

As is known in the art, the electrical connection pad(s) 212, 222, 232, 242 are generally adapted for a direct wired electrical connection to a suitable monitor or sensor electronics device 100 for monitoring a user's condition in response to signals derived from the sensor electrodes 210, 220, 230, 240. Further description of flexible thin film sensors of this general type may be found, e.g., in U.S. Pat. No. 5,391,250, which is herein incorporated by reference. The connection portion 24 may be conveniently connected electrically to the monitor or sensor electronics device 100 or by a connector block 28 (or the like) as shown and described, e.g., in U.S. Pat. No. 5,482,473, which is also herein incorporated by reference. Thus, in accordance with aspects of the present disclosure, subcutaneous sensor sets 10 may be configured or formed to work with either a wired or a wireless characteristic monitor system.

The sensor electrodes 210, 220, 230, 240 may be used in a variety of sensing applications and may be configured in a variety of ways. For example, the sensor electrodes 210, 220, 230, 240 may be used in physiological parameter sensing applications in which some type of biomolecule is used as a catalytic agent. As another example, the sensor electrodes 210, 220, 230, 240 may be used in an oxygen-independent glucose sensor.

The sensor electrodes 210, 220, 230, 240, along with a biomolecule or some other catalytic agent, may be placed in a human body in a vascular or non-vascular environment. For example, the sensor electrodes 210, 220, 230, and 240, and biomolecule may be placed in a vein and be subjected to a bloodstream or may be placed in a subcutaneous or peritoneal region of the human body.

The monitor 100 may also be referred to as a sensor electronics device 100. The monitor 100 may include a power source 110, a sensor interface 122, processing electronics 124, and data formatting electronics 128. The monitor 100 may be coupled to the sensor set 10 by a cable 102 through a connector that is electrically coupled to the connector block 28 of the connection portion 24. In an alternative aspect, the cable 102 may be omitted. In this aspect of the disclosure, the monitor 100 may include an appropriate connector for direct connection to the connection portion 104 (FIG. 1) of the sensor set 10. The sensor set 10 may be modified to have the connector portion 104 positioned at a different location, e.g., on top of the sensor set 10 to facilitate placement of the monitor 100 over the sensor set 10.

In aspects of the disclosure, the sensor interface 122, the processing electronics 124, and the data formatting electronics 128 are formed as separate semiconductor chips. However, alternative aspects may combine the various semiconductor chips into a single or multiple customized semiconductor chips. The sensor interface 122 connects with the cable 102 that is connected with the sensor set 10. The processing electronics 124 may include a processor 129 and memory 130. The memory 130 stores instructions which, when executed by the processor, cause the sensor electronics device 100 to perform various functions, such as process the sensor signals.

The processor 129 receives the sensor signal (e.g., a measured current or voltage) after the sensor signal is measured at the analyte sensor (e.g., the working electrode). The processor 129 processes the sensor signal and generates a processed sensor signal. The processor 129 calibrates the processed sensor signal utilizing reference values. In an aspect of the disclosure, the reference values are stored in a reference memory. The processor 129 generates sensor measurements. The sensor measurements may be stored in the memory 130. The sensor measurements may be sent to a display/transmission device to be either displayed on a display in a housing with the sensor electronics or transmitted to an external device.

The sensor electronics device 100 may be a monitor which includes a display-to-display physiological characteristics readings. The sensor electronics device 100 may also be installed in a desktop computer, a pager, a television including communications capabilities, a laptop computer, a server, a network computer, a personal digital assistant (PDA), a portable telephone including computer functions, an infusion pump including a display, and/or a combination infusion pump/analyte sensor. The sensor electronics device 100 may be housed in a cellular phone, a smartphone, a network device, a home network device, and/or other appliance connected to a home network.

The power source 110 may be a battery. The battery can include three series silver oxide battery cells. In alternative aspects, different battery chemistries may be utilized, such as lithium based chemistries, alkaline batteries, nickel metal hydride, or the like, and a different number of batteries may be used. The monitor 100 provides power to the sensor set via the power source 110, through the cable 102 and cable connector 104. In an aspect of the disclosure, the power is a voltage provided to the sensor set 10. In an aspect of the disclosure, the power is a current provided to the sensor set 10. In an aspect of the disclosure, the power is a voltage provided at a specific voltage to the sensor set 10.

FIGS. 2 and 3 illustrate an implantable multi-electrode sensor array 200 according to an aspect of the present disclosure. The multi-electrode sensor array 200 generally includes a plurality of electrodes, for example, a first electrode 210, a second electrode 220, a third electrode 230, and a fourth electrode 240. Although the above examples use four electrodes, any number of electrodes, more than two, may be used. Each of the electrodes 210, 220, 230, 240 of the multi-electrode sensor array 200, may be separately fabricated using processes that would be incompatible with processes of the other electrodes, e.g., from a separate wafer, so that the incompatible processes do not cause contamination.

Each electrode 210-240 of the plurality of electrodes may be configured for sensing a different analyte. For example, in an example configuration, the first working electrode 210 may be configured to sense ketones, the second electrode 220 may be configured to act as a counter electrode, the third electrode 230 may be configured to act as a reference electrode, and the fourth working electrode 240 may be configured to sense glucose. Although the above analytes are used as an example, any number of analytes may be tested for.

Each of the working electrodes 210, 220, 230, and 240, may include an electrical connection 212, 242, and/or active portion 214, 234, 244 disposed on either a first side or a second side of a substrate.

The first electrode 210 includes a first surface 215 and a second surface 216. The first surface 215 of the first electrode 210 includes an electrical connection pad 212 on a first end portion of the first electrode 210, and a first enzyme on an active portion 214 of the first electrode 210. The first enzyme is configured for sensing the first analyte (e.g., a ketone). It is contemplated that an electrode can have more than one sensing area (e.g., more than one enzyme area).

The second electrode 220 includes a first surface 225 and a second surface 226. The first surface 225 of the second electrode 220 (e.g., a counter electrode) is bonded to a second surface 216 of the first electrode 210. The electrodes may be bonded together using any suitable method. On each end of the second electrode 220, a first portion 227 of the second electrode extends past the first electrode 210, exposing the electrical connection pad 222 or the active portion (not shown) of the second electrode 220. Thus, enabling electrical connection to the electrical connection pad 222 of the second electrode 220 which is adapted for a direct wired electrical connection to a suitable monitor or sensor electronics device 100 (FIG. 1) for monitoring a user's condition in response to signals derived from the second electrode 220. For example, about 600 microns to about 800 microns of the second electrode may be exposed on either end portion of the second electrode 220.

The third electrode 230 includes a first surface 235 and a second surface 236. The second surface 236 of the third electrode 230 (e.g., a reference electrode) is bonded to a second surface 226 of the second electrode 220. Although a reference electrode is used as an example, the third electrode may be configured to include an active portion to sense an analyte different from the first analyte.

The fourth electrode 240 includes a first surface 245 and a second surface 246. The second surface 246 of the fourth electrode 240 is bonded to the first surface 235 of the third electrode 230. The first surface 245 of the fourth electrode 240 includes a first electrical connection pad 242 on a first end portion of the fourth electrode 240, and a first enzyme on an active portion 244 of the first electrode 240, the first enzyme configured for sensing the first analyte (e.g., GOx).

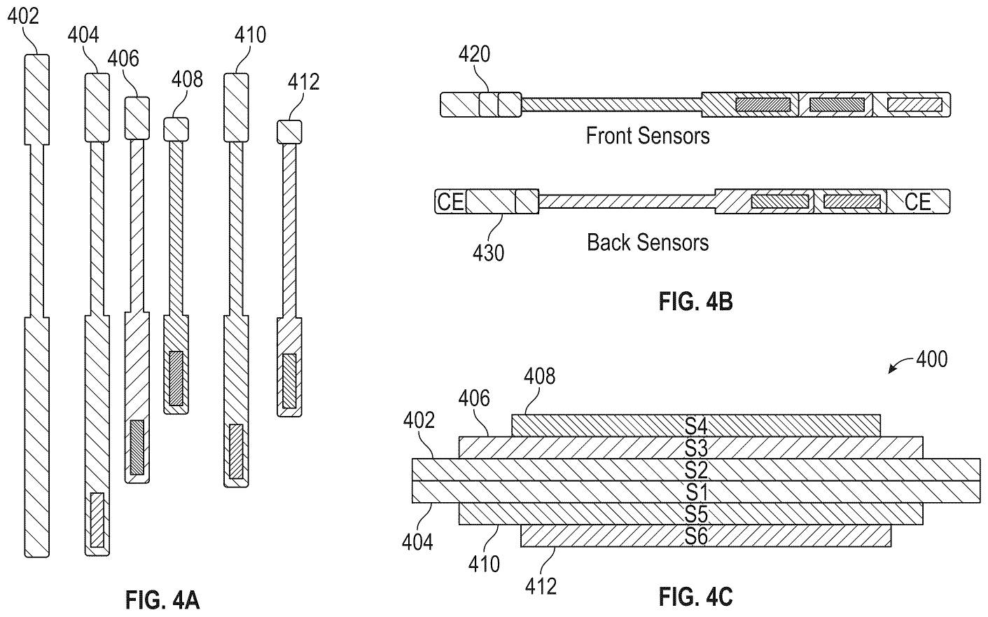

FIGS. 4A-4C illustrate an alternative aspect, including an example six-electrode multi-electrode sensor array 400 of the sensor insert set of FIG. 1. The multi-electrode sensor array 400 of FIGS. 4A-4C are similar to the multi-electrode sensor array 200 of FIGS. 2 and 3, and the differences will be described below.

The multi-electrode sensor array 400 includes a front stack 420 of sensors bonded to a back stack 430 of sensors (FIG. 4B). The front stack 420 includes a first electrode 408 configured for sensing a second analyte, a second electrode 406 configured for sensing a first analyte, and a third electrode 402 configured as a counter electrode (or for sensing a third analyte). The back stack 430 includes a fourth electrode 404 configured for sensing a fourth analyte, a fifth electrode 410 configured for sensing a fifth analyte, and the sixth electrode 412 configured as a reference electrode (or for sensing a sixth analyte). It is contemplated that the counter electrode and/or reference electrode could be in any suitable position in the front stack 420 or the back stack 430. For example, the counter electrode may be the second electrode 406, and the reference electrode mat be the third electrode 402.

For example, for a multi-electrode sensor array 400 configured for diabetes care, the first electrode 408 may be configured for sensing sodium, the second electrode 406 may be configured for sensing a ketone, and the third electrode 402 may be configured as a counter electrode. The fourth electrode 404 may be configured for sensing glucose, the fifth electrode 410 may be configured for sensing potassium, and the sixth electrode 412 may be configured as a reference electrode.

In another example, for a multi-electrode sensor array 400 configured for acute kidney injury (AKI) and/or congestive heart failure (CHF) monitoring, the first electrode 408 may be configured for sensing sodium, the second electrode 406 may be configured for sensing creatine, and the third electrode 402 may be configured as a counter electrode. The fourth electrode 404 may be configured for sensing glucose, the fifth electrode 410 may be configured for sensing potassium, and the sixth electrode 412 may be configured as a reference electrode.

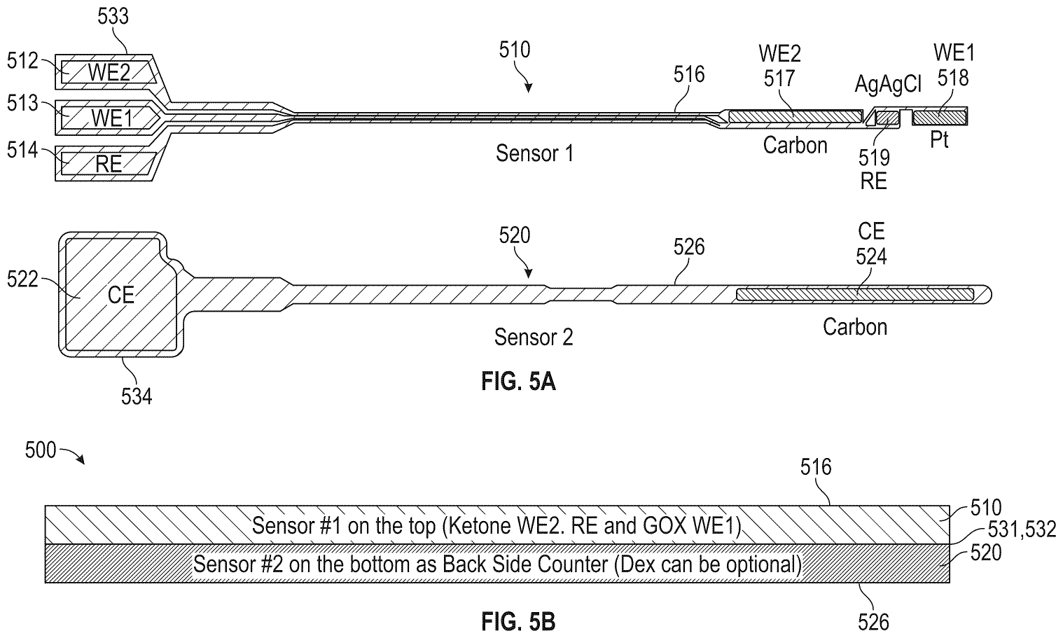

FIGS. 5A and 5B illustrate an alternative aspect, including an example four-electrode multi-electrode sensor array 500 of the sensor insert set of FIG. 1. The four-electrode multi-electrode sensor array 500 includes a first sensor 510 configured to sense a first analyte bonded to a second sensor 520 configured to sense a second analyte different from the first analyte. The disclosed construction of the four-electrode multi-electrode sensor array 500 enables the manufacture of the four-electrode multi-electrode sensor array 500 using incompatible fabrication processes that would otherwise cause contamination of the sensor's active portions. An incompatible process is a process performed to manufacture one electrode that would interfere with the manufacture of another electrode. It is contemplated that the multi-electrode sensor array 500 may be fabricated with each of the electrodes using compatible processes. For example, the first sensor 510 may be configured to test ketone, to test glucose, and as a reference electrode, while the second sensor 520 may be configured as a counter electrode (FIG. 5B).

The first sensor 510 includes a first surface 516 and a second surface 531. The first surface 516 of the first sensor 510 includes a first sensing region (e.g., enzyme bound to carbon) disposed on a first active portion 517, a second sensing region (e.g., enzyme bound to platinum) on a second active portion 518, and third sensing region (e.g., bound to silver chloride) disposed on a third active portion 519 of the first sensor 510. The first sensor 510 further includes a first electrical connection pad 513 (i.e., WE1), a second electrical connection pad 512 (i.e., WE2), and an electrical connection pad electrode 514 (i.e., RE) on a first end portion 533 of the first sensor 510. The second sensor 520 includes a first surface 526 and a second surface 532. The first surface 526 of the second sensor 520 includes a first sensing region (e.g., ionophore bound to carbon) disposed on a first active portion 524, and a fourth electrical connection pad 522 (i.e., CE) disposed on a first end portion 534 of the first sensor 510. The second surface 531 of the first sensor 510 is bonded to the second surface 532 of the second sensor 520.

For example, the four-electrode multi-electrode sensor array 500 may be configured as a “Dual Sensor” (e.g., sensing a ketone and glucose). For example, the first sensor 510 may be configured to sense glucose and the second sensor 520 may be configured to sense creatine and potassium. One of skill in the art would be familiar with how to implement an electrode that is configured to sense creatine and separately an electrode configured to sense potassium.

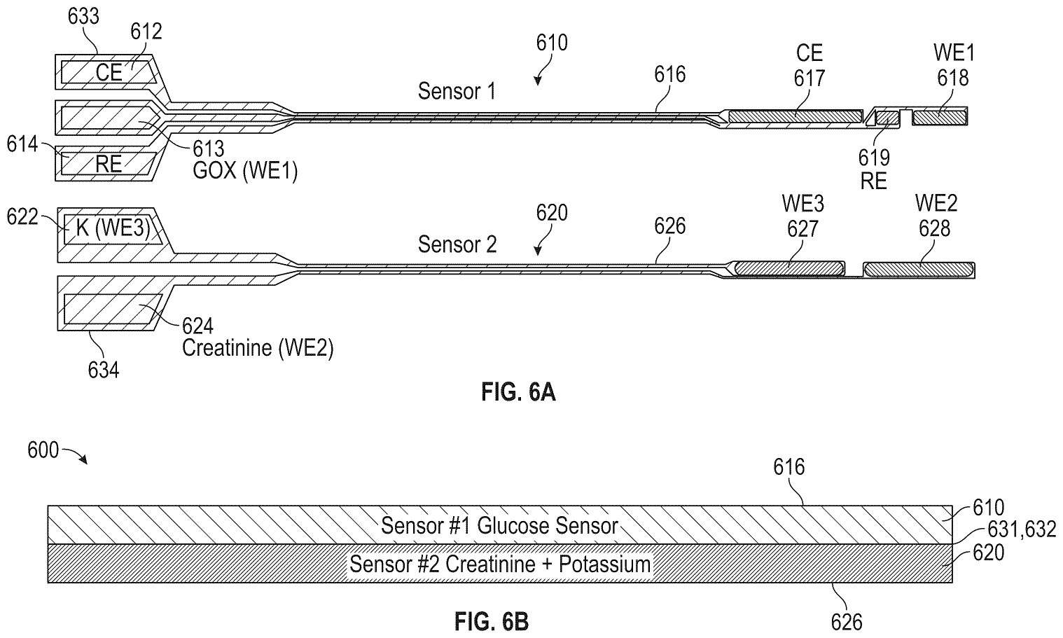

FIGS. 6A and 6B illustrate an alternative aspect, including an example five-electrode multi-electrode sensor array 600 of the sensor insert set of FIG. 1. The five-electrode multi-electrode sensor array 600 includes a first sensor 610 configured to sense a first analyte bonded to a second sensor 620 configured to sense a second and third analyte, which are different from the first analyte. In aspects, the third analyte may be different from the first and second analytes. This provides the benefit of enabling a customized sensor for disease management for a given patient's condition. For example, for acute kidney failure the health care provider may only need to monitor glucose, creatinine, and potassium.

The first electrode 610 includes a first surface 616 and a second surface 631. The first surface 616 of the first electrode 610 includes a first enzyme disposed on a first active portion 617, a second enzyme on a second active portion 618, and third enzyme disposed on a third active portion 619 of the first sensor 610. The first sensor 610 further includes a first electrical connection pad 613 (i.e., WE1), a second electrical connection pad 612 (i.e., WE2), and an electrical connection pad electrode 614 (i.e., RE) on a first end portion 633 of the first sensor 610.

The second sensor 620 includes a first surface 626 and a second surface 632. The first surface 626 of the second sensor 620 includes a first enzyme disposed on a first active portion 627, a first enzyme disposed on a second active portion 628, a fourth electrical connection pad 622 (i.e., WE3) and a fifth electrical connection 624 both disposed on a first end portion 634 of the second sensor 620. The second surface 631 of the first sensor 610 is bonded to the second surface 632 of the second sensor 620.

For example, the five-electrode multi-electrode sensor array 600 may be configured as a “Tri Sensor” (e.g., sensing creatine, potassium, and glucose). For example, the first sensor 610 may be configured to sense glucose and the second sensor 620 may be configured to sense creatine and potassium. Persons skilled in the art know how to implement sensors for sensing creatine, potassium, and glucose.

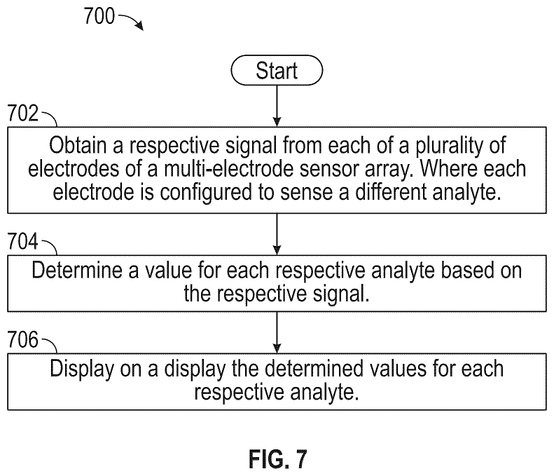

Referring to FIG. 7, a processor-implemented method 700 for using the multi-electrode sensor array of any of FIGS. 2-6B, using the sensor set 10 of FIG. 1 is shown. The sensor set 10 for using the multi-electrode sensor array may include processor 129 and memory 130 (FIG. 1) including instructions stored thereon which, when executed by processor 129, cause the sensor set 10 to perform the steps of method 700.

Initially, at step 702, the processor 129 causes the sensor electronics device 100 to obtain a respective signal from a plurality of electrodes of the multi-electrode sensor array 200. Although multi-electrode sensor array 200 is used as an example, the method is equally applicable to multi-electrode sensor array 400, 500, 600. Each of the plurality of electrodes of the multi-electrode sensor array is configured to sense a different analyte. The analytes may include, for example, blood urea nitrogen, carbon dioxide, creatinine, glucose, chloride, calcium, potassium, and/or sodium. Persons skilled in the art would understand how to implement sensors for detecting each of blood urea nitrogen, carbon dioxide, creatinine, glucose, chloride, calcium, potassium, and/or sodium. Each electrode of the multi-electrode sensor array 200 creates a sensor signal indicative of a concentration of a physiological characteristic being measured. For example, the sensor signal may be indicative of a blood glucose reading. The sensor signal may be measured at the active portion 214, 234, 244 of a working electrode 210, 230, 240 (FIG. 3). In an aspect of the disclosure, the sensor signal may be a current measured at the active portion 214, 234, 244 (e.g., working electrode). In an aspect of the disclosure, the sensor signal may be a voltage measured at the working electrode.

At step 704, the processor 129 causes the sensor set 10 to determine a value for each respective analyte based on the respective signal. After the physiological characteristic value is determined by the processor 129, the processor 129 may store measurements of the physiological characteristic values for a number of time periods. For example, a blood glucose value (BG) may be sent to the processor 129 from the sensor every second or five seconds, and the processor may save sensor measurements for five minutes or ten minutes of BG readings. The processor 129 may transfer the measurements of the physiological characteristic values to a display on the sensor electronics device 100. For example, the sensor electronics device 100 may be a monitor which includes a display that provides a blood glucose reading for a subject. In one aspect of the disclosure, the processor 129 may transfer the measurements of the physiological characteristic values to an output interface of the processor 129. The output interface of the processor 129 may transfer the measurements of the physiological characteristic values, e.g., blood glucose values, to an external device, e.g., an infusion pump, a combined infusion pump/glucose meter, a computer, a personal digital assistant, a pager, a network appliance, a server, a cellular phone, or any computing device.

At step 706, the processor 129 causes the sensor set 10 to display, on a screen, the determined values for each respective analyte. In aspects, the sensor set 10 is configured to enable the capturing of a basic metabolic panel (e.g., Chem-7 or Chem 8). The basic metabolic panel generally measures the levels of blood urea nitrogen (BUN), carbon dioxide, creatinine, glucose, chloride, calcium, potassium, and/or sodium, in the user (e.g., the patient). Although enzymatic sensors are used as an example, it is contemplated that nonenzymatic sensors such as potentiometric sensors, aptamer-based sensors may be used. For example, the electrodes may be stacked according to enzymatic and non-enzymatic phenotypes, which could facilitate integration of these different sensor modalities.

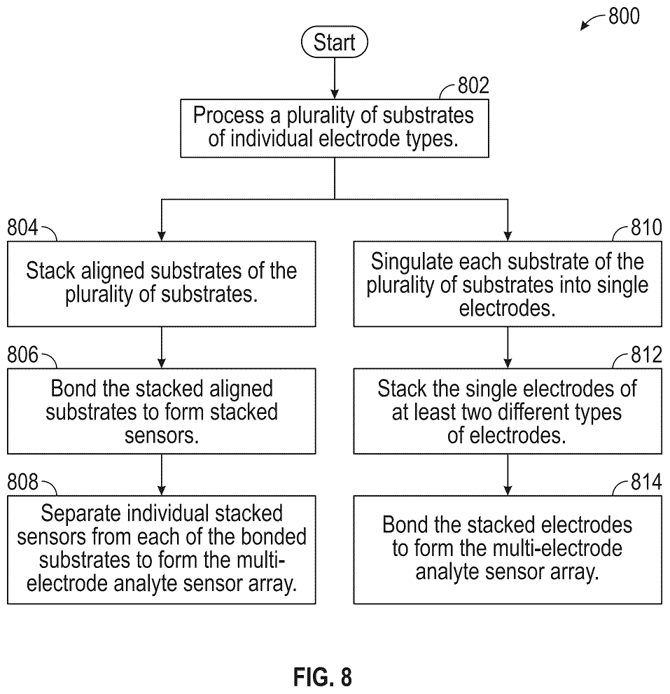

Referring to FIG. 8, a method 800 of manufacturing a multi-electrode analyte sensor array 200, 400, 500, 600 of FIGS. 2-6B, is shown.

At step 802, a plurality of substrates of individual electrode types is processed. In aspects, each of the plurality of substrates includes only the same electrode type on that wafer. The electrodes 210, 220, 230, 240 are constructed in accordance with thin film mask techniques to include elongated thin film conductors embedded or encased between layers of a selected insulative material such as polyimide film or sheet, and membrane. The sensing portion 214 (e.g., the enzyme) and the electrical connection pad 212 will be exposed (FIG. 3).

In aspects, the plurality of substrates includes at least two or more different electrode types. For example, the plurality of substrates each includes a first electrode and a second electrode from different substrates. The first electrode is configured for sensing the first analyte. The first surface of the first electrode includes a first electrical connection pad on a first end portion of the first electrode, and a first sensing region on a second portion of the first electrode. The first enzyme is configured for sensing the first analyte. The second electrode is configured for sensing a second analyte. The first surface of the second electrode includes a second electrical connection pad on the first end portion of the second electrode and a second enzyme on the second portion of the second electrode. The second enzyme is configured for sensing the second analyte. Although two electrodes are used as an example, any number of suitable electrodes may be used, for example, 4 or 20 electrodes.

The multi-electrode analyte sensor array may be formed by either stacking substrates and then separating the individual stacked sensors (see steps 804-808) or by singulating the electrodes and then stacking the single electrodes (see steps 810-814).

At step 804 aligned substrates of the plurality of substrates are stacked. At step 806 the stacked aligned substrates are bonded to form stacked sensors. Bonding may include any suitable method of bonding, for example, UV curing. At step 808 the individual stacked sensors are separated from each of the bonded substrates to form the multi-electrode analyte sensor array 200.

At step 810 each wafer of the plurality of substrates is singulated into single electrodes. At step 812 the single electrodes of at least two different types of electrodes are stacked. For example, a first type may be an electrode configured to sense glucose and a second type may be an electrode configured to sense potassium. At step 814, the stacked electrodes are bonded to form the multi-electrode analyte sensor array. In aspects, the multi-electrode analyte sensor array may be a flex stack array. In aspects, the stacked array could be one sided, two sided, or multi-sided. For example, a Chem-8 cBMP could be constructed on an octagon cross-sectioned “needle” assembly. In aspects, the electrodes may be stacked according to enzymatic and non-enzymatic phenotypes which could facilitate integration of these different sensor modalities.

In aspects, the electrodes may be individually calibrated prior to stacking and/or bonding.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, the above-described servers and computing devices.

While the description above refers to particular aspects of the present disclosure, it will be understood that many modifications may be made without departing from the spirit thereof. Additional steps and changes to the order of the algorithms can be made while still performing the key teachings of the present disclosure. Thus, the accompanying claims are intended to cover such modifications as would fall within the true scope and spirit of the present disclosure. The presently disclosed aspects are, therefore, to be considered in all respects as illustrative and not restrictive, the scope of the disclosure being indicated by the appended claims rather than the foregoing description. Unless the context indicates otherwise, any aspect disclosed herein may be combined with any other aspect or aspects disclosed herein. All changes that come within the meaning of, and range of, equivalency of the claims are intended to be embraced therein.

What is claimed is:

1. A multi-electrode sensor array comprising:
a first electrode configured for sensing a first analyte, the first electrode comprising:
a first surface of the first electrode which includes a first electrical connection pad on a first end portion of the first electrode, and
a first sensing region on a second portion of the first electrode, the first enzyme configured for sensing the first analyte with enzyme; and
a second electrode configured for sensing a second analyte, the second electrode comprising:
a first surface of the second electrode which includes a second electrical connection pad on a first end portion of the second electrode, and
a second enzyme on a second portion of the second electrode, the second enzyme configured for sensing the second analyte.

2. The multi-electrode sensor array of claim 1, wherein the first analyte is different from the second analyte.

3. The multi-electrode sensor array of claim 1, wherein the second electrode is bonded to a second surface of the first electrode.

4. The multi-electrode sensor array of claim 3, wherein the first sensing region includes a first analyte sensing molecule.

5. The multi-electrode sensor array of claim 4, wherein the first electrode is electrografted or electropolymerized with the first analyte sensing molecule.

6. The multi-electrode sensor array of claim 1, wherein the first and second electrodes are each configured to generate a detectable electrical signal upon exposure to the respective first and second analyte.

7. The multi-electrode sensor array of claim 1, wherein the first electrode and the second electrode are formed by incompatible manufacturing processes.

8. The multi-electrode sensor array of claim 1, further comprising:
a third electrode configured for sensing a third analyte, the third electrode comprising:
a first surface of the third electrode which includes a third electrical connection pad on a first end portion of the third electrode, and
a third enzyme on a second portion of the third electrode, the third enzyme configured for sensing the third analyte.

9. The multi-electrode sensor array of claim 8, wherein the third analyte is different from the first and second analytes.

10. The multi-electrode sensor array of claim 8, wherein a second surface of the second electrode is bonded to a second surface of the third electrode.

11. The multi-electrode sensor array of claim 1, wherein the first end portion of the second electrode and the second portion of the second electrode extend past the first end portion of the first electrode and the second portion of the first electrode, respectively.

12. The multi-electrode sensor array of claim 1, wherein the first electrode further comprises:
a third electrical connection pad on the first end portion of the first electrode; and
a third enzyme on a second portion of the first electrode, the third enzyme configured for sensing a third analyte.

13. A method of manufacturing a multi-electrode analyte sensor array, the method comprising:
processing a plurality of substrates of individual electrode types, including a first electrode and a second electrode from different substrates,
wherein the first electrode is configured for sensing a first analyte, a first surface of the first electrode includes a first electrical connection pad on a first end portion of the first electrode, and a first enzyme on a second portion of the first electrode, the first enzyme configured for sensing the first analyte; and
wherein the second electrode is configured for sensing a second analyte, a first surface of the second electrode includes a second electrical connection pad on a first end portion of the second electrode and a second enzyme on a second portion of the second electrode, the second enzyme configured for sensing the second analyte.

14. The method of claim 13, further comprising:
stacking aligned substrates of the plurality of substrates;
bonding the stacked aligned substrates to form stacked sensors; and
separating individual stacked sensors from each of the bonded substrates to form the multi-electrode analyte sensor array.

15. The method of claim 13, further comprising:
singulating each wafer of the plurality of substrates into single electrodes;
stacking the single electrodes of at least two different types of electrodes; and
bonding the stacked electrodes to form the multi-electrode analyte sensor array,
wherein the multi-electrode analyte sensor array is a flex stack array.

16. The method of claim 13, wherein the plurality of substrates of individual electrode types further comprise a third electrode configured for sensing a third analyte, a first surface of the third electrode includes a third electrical connection pad on a first end portion of the third electrode and a third enzyme on a second portion of the third electrode, the third enzyme configured for sensing the third analyte.

17. The method of claim 13, wherein each of the plurality of substrates includes only the same electrode type on that wafer.

18. The method of claim 13, wherein the plurality of substrates includes at least two or more different electrode types.

19. The method of claim 13, further comprising stacking a third electrode to the multi-electrode analyte sensor array, the third electrode configured for sensing a third analyte, a first surface of the third electrode includes a third electrical connection pad on a first end portion of the third electrode and a third enzyme on a second portion of the third electrode, the third enzyme configured for sensing the third analyte.

20. A system for sensing multiple analytes, the system comprising:
a multi-electrode sensor array comprising:
a first electrode configured for sensing a first analyte, a first surface of the first electrode includes a first electrical connection pad on a first end portion of the first electrode, and a first enzyme on a second portion of the first electrode, the first enzyme configured for sensing the first analyte; and
a second electrode configured for sensing a second analyte, a first surface of the second electrode includes a second electrical connection pad on a first end portion of the second electrode and a second enzyme on a second portion of the second electrode, the second enzyme configured for sensing the second analyte;
a processor wherein the first electrode and second electrode are communicatively coupled to the processor; and
a memory, including instructions stored thereon which, when executed by the processor, cause the system to:
obtain a respective signal from each of the first electrode and the second electrode;
determine a value for the first analyte based on the signal from the first electrode;
determine a value for the second analyte based on the signal from the second electrode; and
display on a display the determined values for the first analyte and the second analyte.
